(19) United States
(12) Patent Application Publication
(10) Pub. No.: US 2026/0354886 A1
(43) Pub. Date: Nov. 12, 2026

(54) TISSUE TREATING ELECTRODES FOR SURGICAL INSTRUMENTS AND SURGICAL INSTRUMENTS INCORPORATING THE SAME

(71) Applicant: Covidien LP, Mansfield, MA (US)

(72) Inventors: Chelsea E. WALBRIDGE, Longmont, CO (US); Prakash MANLEY, Arvada, CO (US); Xiaoming CHENG, Marana, AZ (US); Daniel W. MERCIER, Erie, CO (US); Sean W. MILLER, Broomfield, CO (US); Craig V. KRASTINS, Arvada, CO (US); Jake H. YOUNG, Erie, CO (US)

(21) Appl. No.: 19/477,153

(22) PCT Filed: Apr. 24, 2024
(86) PCT No.: PCT/US2024/026059

§ 371 Date: Oct. 21, 2025

Related U.S. Application Data
(60) Provisional application No. 63/461,992, filed on Apr. 26, 2023.

(51) Int. Cl. A61B 18/14; A61B 18/00

(52) U.S. Cl. CPC A61B 18/1445; A61B 2018/00083; A61B 2018/00107; A61B 2018/1452

(57) ABSTRACT

An end effector assembly includes first and second jaw members and an electrode. One or both of the jaw members is movable relative to the other from a spaced-apart position to an approximated position to grasp tissue. The electrode is supported by, extends longitudinally along, and protrudes from the second jaw member. The electrode includes an electrically conductive element and an electrically insulative coating. The electrically conductive element defines an elongate base surface, first and second elongate side surfaces, a proximal end portion, and a distal end portion covered by the electrically insulative coating. The electrically conductive element further defines an elongate tissue treating surface opposing the elongate base surface that is left exposed such that energizing the electrically conductive element with RF energy concentrates the RF energy at the exposed elongate tissue treating surface.

### CROSS-REFERENCE TO RELATED APPLICATIONS

This application claims the benefit of, and priority to, U.S. Provisional Patent Application No. 63/461,992, filed on Apr. 26, 2023, the entire contents of which are hereby incorporated herein by reference.

### FIELD

The present disclosure relates to surgical instruments and, more particularly, to tissue treating electrodes for surgical instruments and surgical instruments incorporating the same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

Energy-based elements are also utilized in various other surgical instruments and/or to otherwise facilitate treating tissue, e.g., coagulating tissue, sealing tissue, cutting tissue, etc., using energy, e.g., thermal, electrosurgical, ultrasonic, light, or other suitable energy.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with aspects of the present disclosure is an end effector assembly of a surgical instrument. The end effector assembly includes first and second jaw members and an electrode. Each of the first and second jaw members defines a tissue treating surface. At least one of the first jaw member or the second jaw member is movable relative to another of the first jaw member or the second jaw member from a spaced-apart position to an approximated position to grasp tissue between the tissue treating surfaces of the first and second jaw members. The electrode is supported by the second jaw member, extends longitudinally along at least a portion of a length of the second jaw member, and protrudes from the second jaw member towards the first jaw member. The electrode includes an electrically conductive element and an electrically insulative coating. The electrically conductive element defines an elongate base surface, an elongate tissue treating surface opposing the elongate base surface, first and second elongate side surfaces, a proximal end portion, and a distal end portion. The electrically insulative coating covers the elongate base surface, the first and second elongate side surfaces, the proximal end portion, and the distal end portion, leaving the elongate tissue treating surface exposed. The electrically conductive element is adapted to connect to a source of Radio Frequency (RF) energy for energizing the electrically conductive element with RF energy and concentrating the RF energy at the exposed elongate tissue treating surface.

In an aspect of the present disclosure, the electrode defines a width at the elongate tissue treating surface of from about 0.001 inches to about 0.002 inches.

In another aspect of the present disclosure, a thickness of the electrically insulative coating on each of the first and second elongate side surfaces tapers in a direction from the elongate base surface to the elongate tissue treating surface.

In still another aspect of the present disclosure, a maximum thickness of the electrically insulative coating on each of the first and second elongate side surfaces is from about 0.002 inches to about 0.006 inches.

In yet another aspect of the present disclosure, the electrode protrudes from the tissue treating surface of the second jaw member towards the first jaw member a height of from about 0.018 inches to about 0.024 inches.

In still yet another aspect of the present disclosure, the electrode includes an exposed portion of the electrically conductive element at or towards the proximal end portion of the electrically conductive element for connection of an electrical lead wire to the electrically conductive element to provide the RF energy to the electrically conductive element.

In another aspect of the present disclosure, the electrode includes a body portion and a tapered distal portion extending distally from the body portion to a distal tip of the electrode.

In still another aspect of the present disclosure, the electrode tapers in width along at least a portion of a height of the electrode extending from the elongate base surface to the elongate tissue treating surface.

In yet another aspect of the present disclosure, the electrode defines a transition at the interface between the elongate tissue treating surface and at least one of the proximal end portion or the distal end portion.

In another aspect of the present disclosure, the electrically insulative coating is glass.

In still another aspect of the present disclosure, a compliant electrically insulative member is disposed within the first jaw member and positioned to oppose the electrode in the approximated position. In such aspects, the compliant electrically insulative member may define an exposed surface that is substantially co-planar with the tissue treating surface of the first jaw member.

In yet another aspect of the present disclosure, the compliant electrically insulative member defines a durometer of from about 55 D to about 65 D. Additionally or alternatively, the compliant electrically insulative member may define a width of from about 0.030 inches to about 0.040 inches and/or a depth of from about 0.035 inches to about 0.045 inches.

In still yet another aspect of the present disclosure, the tissue treating surfaces of the first and second jaw members are adapted to connect to a source of RF energy for energizing the tissue treating surfaces of the first and second jaw members with RF energy at different potentials.

In another aspect of the present disclosure, one of the first jaw member or the second jaw member is fixed and another of the first jaw member or the second jaw member is movable relative to the fixed jaw member.

Another end effector assembly of a surgical instrument provided in accordance with the present disclosure includes first and second jaw members each defining a tissue treating surface. At least one of the first jaw member or the second jaw member is movable relative to another of the first jaw member or the second jaw member from a spaced-apart position to an approximate position to grasp tissue between the tissue treating surfaces of the first and second jaw members. An electrode is supported by the second jaw member, extends longitudinally along at least a portion of a length of the second jaw member between proximal and distal ends of the electrode, and protrudes from the second jaw member towards the first jaw member to an elongate upper extent of the electrode. The electrode is adapted to connect to a source of Radio Frequency (RF) energy for energizing the electrode with RF energy. The electrode defines a first transition between the elongate upper extent of the electrode and the proximal end of the electrode and a second transition between the elongate upper extent of the electrode and the distal end of the electrode.

In an aspect of the present disclosure, the first transition is more gradual than the second transition.

In another aspect of the present disclosure, the first transition defines a first radius of curvature and wherein the second transition defines a second radius of curvature less than the first radius of curvature.

In still another aspect of the present disclosure, the electrode includes an electrically conductive element and an electrically insulative coating. The electrically insulative coating substantially covers the electrically conductive element except along the elongate upper extent of the electrode such that the electrically conductive element is exposed along the elongate upper extent of the electrode.

In yet another aspect of the present disclosure, the tissue treating surfaces of the first and second jaw members are adapted to connect to a source of RF energy for energizing the tissue treating surfaces of the first and second jaw members with RF energy at different potentials.

In still yet another aspect of the present disclosure, one of the first jaw member or the second jaw member is fixed and another of the first jaw member or the second jaw member is movable relative to the fixed jaw member.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of an end effector assembly of the forceps of FIG. 1 including first and second jaw members;

FIG. 5A is a perspective view of the first jaw member of the end effector assembly of the forceps of FIG. 1;

FIG. 5B is a perspective view of the second jaw member of the end effector assembly of the forceps of FIG. 1;

FIG. 6 is a transverse, cross-sectional view of one of the jaw members of the end effector assembly of the forceps of FIG. 1 including a tissue treating electrode in accordance with the present disclosure;

FIG. 7 is a perspective view of the tissue treating electrode of FIG. 6 including an electrical connector attached thereto;

FIG. 8 is a top view of a distal portion of another tissue treating electrode in accordance with the present disclosure and configured for use with one of the jaw members of the end effector assembly of the forceps of FIG. 1;

FIGS. 9A and 9B are side views of a portion of the end effector assembly of the forceps of FIG. 1 including another tissue treating electrode in accordance with the present disclosure, wherein the jaw members of the end effector assembly are disposed in spaced apart and approximated positions, respectively;

FIGS. 9C-9E illustrate transition configurations in accordance with the present disclosure for use with the tissue treating electrodes of the present disclosure or any other suitable tissue treating electrode;

FIG. 10A is a transverse, cross-sectional view of the end effector assembly of the forceps of FIG. 1 wherein one of the jaw members includes a tissue treating electrode and the other jaw member includes a compliant member opposing the tissue treating electrode; and

FIG. 10B is a transverse, cross-sectional view of the jaw member including the compliant member as shown in FIG. 10A.

### DETAILED DESCRIPTION

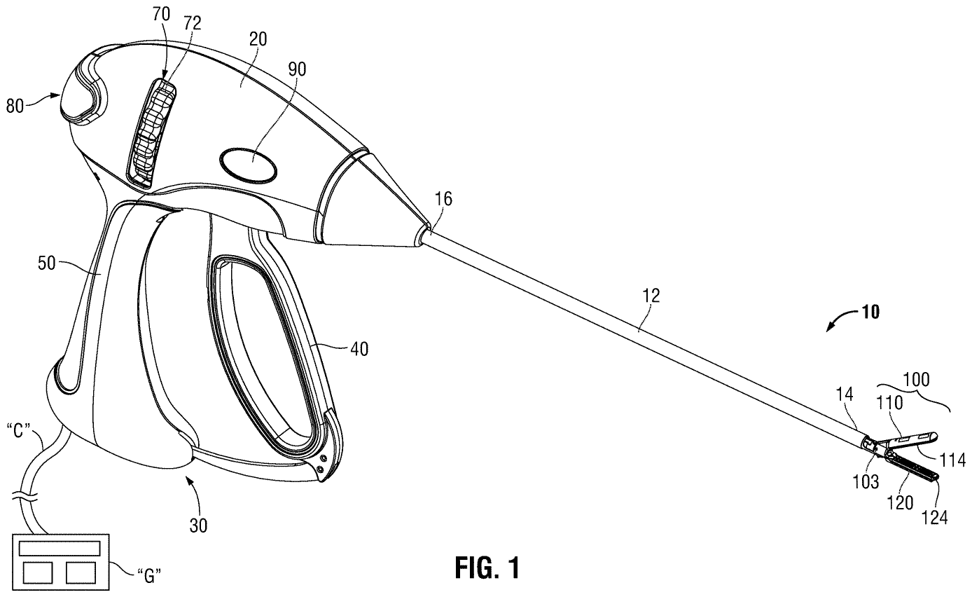

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

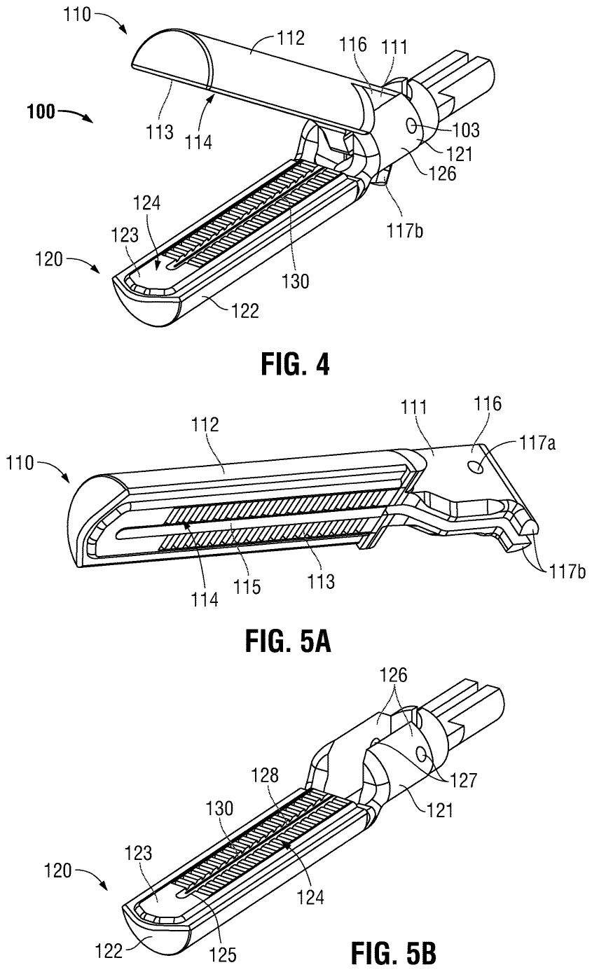

Forceps 10 includes a housing 20, a handle assembly 30, a rotating assembly 70, a first activation switch 80, a second activation switch 90, and an end effector assembly 100. Forceps 10 further includes a shaft 12 having a distal end portion 14 configured to (directly or indirectly) engage end effector assembly 100 and a proximal end portion 16 that (directly or indirectly) engages housing 20. Forceps 10 also includes a cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue treating surfaces 114, 124 of jaw members 110, 120, respectively, of end effector assembly 100 to provide energy thereto. First activation switch 80 is coupled to tissue treating surfaces 114, 124 and the electrosurgical generator “G” to enable the selective activation of the supply of energy to jaw members 110, 120 for treating tissue, e.g., cauterizing, coagulating/desiccating, and/or sealing tissue. Second activation switch 90 is coupled to electrode 130 of jaw member 120 (see FIG. 4) and the electrosurgical generator “G” to enable the selective activation of the supply of energy to electrode 130 (FIG. 4) for treating tissue, e.g., dissecting, spot coagulating, scoring, performing an otomy, etc. As an alternative to first and second activation switches 80, 90, a multi-stage switch may be provided and/or electrosurgical generator “G” may determine the appropriate components to activate (e.g., tissue treating surfaces 114, 124 and/or electrode 130 (FIG. 4)) based upon feedback, sensed properties of forceps 10 and/or tissue, in accordance with a tissue treating algorithm, combinations thereof, or in any other suitable manner.

Rather than providing a separate electrosurgical generator “G,” forceps 10 may be configured as a cordless device such as, for example, including an on-board power source (not shown), e.g., a DC battery, and an on-board electrosurgical generator (not shown) powered by the on-board power source in order to provide energy to electrode 130 (FIG. 4) and/or tissue treating surfaces 114, 124.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

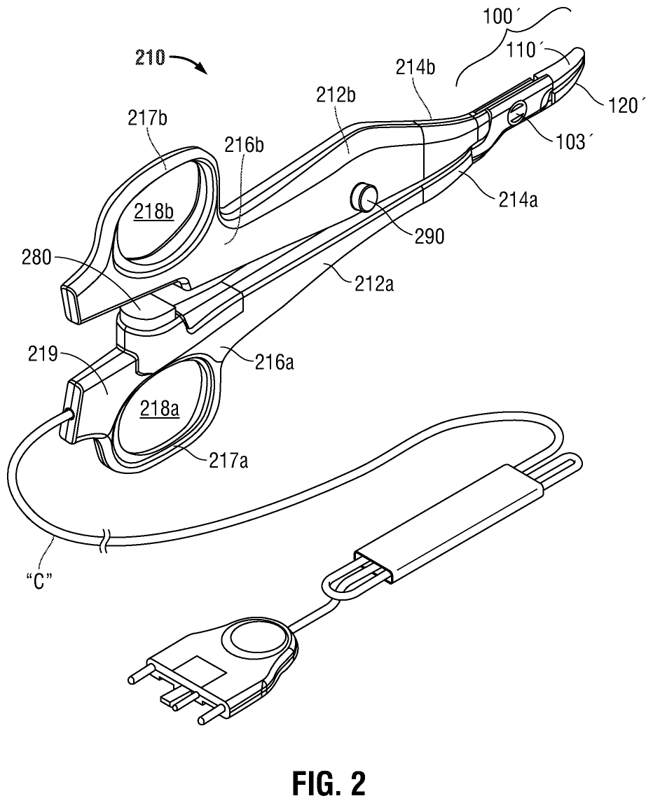

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two shaft members 212a, 212b, each having a proximal end portion 216a, 216b, and a distal end portion 214a, 214b, respectively. Forceps 210 is configured for use with an end effector assembly 100′ similar to end effector assembly 100 (FIGS. 1 and 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 is provided for supplying energy to jaw members 110′, 120′ to treat tissue upon sufficient approximation of shaft members 212a, 212b, e.g., upon activation of first activation switch 280 via shaft member 212a. A second activation switch 290 disposed on either or both of shaft members 212a, 212b is coupled to the electrode (not shown, similar to electrode 130 of jaw member 120 (FIG. 4)) of one of the jaw members 110′, 120′ of end effector assembly 100′ and to the electrosurgical generator “G” for enabling the selective activation of the supply of energy to the electrode for treating tissue. Alternatively, second activation switch 290 may be omitted and the electrosurgical generator “G” (FIG. 1) may be configured to automatically energize the electrode and/or the jaw members 110′, 120′ as appropriate, e.g., based on sensed feedback, according to an algorithm, etc. In particular, electrosurgical generator “G” (FIG. 1) may provide control based on jaw position, handle position, and the like to enable or disable activation of the electrode and/or the jaw members 110′, 120′. Similar functionality may be provided in connection with forceps 10 (FIG. 1).

Jaw members 110′, 120′ define a curved configuration wherein each jaw member 110′, 120′ is similarly curved laterally off of a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

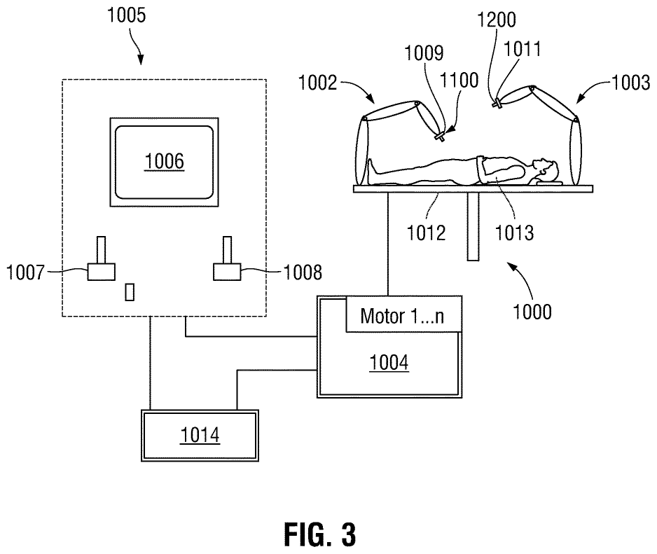

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIG. 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

Turning to FIGS. 4-5B, end effector assembly 100, as noted above, includes first and second jaw members 110, 120. Each jaw member 110, 120 may include a structural frame 111, 121, a jaw housing 112, 122, and a tissue treating plate 113, 123 defining the respective tissue treating surface 114, 124 thereof. Alternatively, only one of the jaw members, e.g., jaw member 120, may include a structural frame 121, jaw housing 122, and tissue treating plate 123 defining the tissue treating surface 124. In such aspects, the other jaw member, e.g., jaw member 110, may be formed as a single unitary body, e.g., a piece of conductive material acting as the structural frame 111 and jaw housing 112 and defining the tissue treating surface 114. An outer surface of the jaw housing 112, in such aspects, may be at least partially coated with an insulative material or may remain exposed. In aspects, tissue treating plates 113, 123 may be deposited onto jaw housings 112, 122 or jaw inserts (not shown) disposed within jaw housings 112, 122, e.g., via sputtering. Alternatively, tissue treating plates 113, 123 may be pre-formed and engaged with jaw housings 112, 122 and/or jaw inserts (not shown) disposed within jaw housings 112, 122 via, for example, overmolding, adhesion, mechanical engagement, etc.

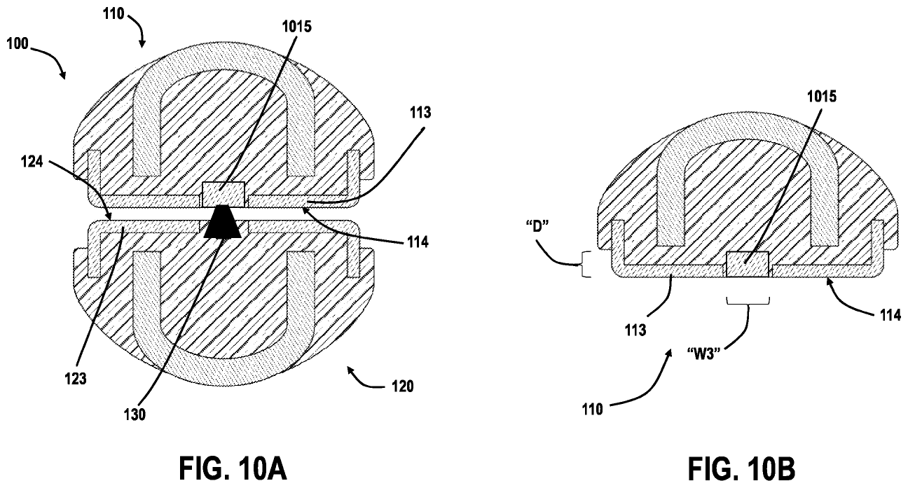

Referring in particular to FIGS. 4 and 5A, jaw member 110, as noted above, may be configured similarly as jaw member 120, may be formed as a single unitary body, or may be formed in any other suitable manner so as to define a structural frame 111 and a tissue treating surface 114 opposing tissue treating surface 124 of jaw member 120. Structural frame 111 includes a proximal flange portion 116 about which jaw member 110 is pivotably coupled to jaw member 120 and a distal body portion (see FIG. 10A) extending distally from proximal flange portion 116. In shaft-based or robotic configurations, proximal flange portion 116 may further include an aperture 117a for receipt of pivot 103 and at least one protrusion 117b extending therefrom that is configured for receipt within an aperture defined within a drive sleeve of the drive assembly (not shown) such that translation of the drive sleeve, e.g., in response to actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a closure tube, a screw-drive mechanism, etc.

Regardless of the particular configuration of jaw member 110, jaw member 110 may include a longitudinally-extending insulative member 115 extending along at least a portion of the length of tissue treating surface 114. Insulative member 115 may be transversely centered on tissue treating surface 114 or may be offset relative thereto. Further, insulative member 115 may be disposed, e.g., deposited, coated, etc., on tissue treating surface 114, may be positioned within a channel or recess defined within tissue treating surface 114, or may define any other suitable configuration. Additionally, insulative member 115 may be substantially (within manufacturing, material, and/or use tolerances) coplanar with tissue treating surface 114, may protrude from tissue treating surface 114, may be recessed relative to tissue treating surface 114, or may include different portions that are coplanar, protruding, and/or recessed relative to tissue treating surface 114. Insulative member 115 may be formed from, for example, thermoplastic elastomer (TPE), silicone, polybenzimidazole, ceramic, parylene, nylon, PTFE, or other suitable material(s) (including combinations of insulative and non-insulative materials).

With reference to FIGS. 4 and 5B, as noted above, jaw member 120 includes a structural frame 121, a jaw housing 122, and a tissue treating plate 123 defining the tissue treating surface 124 thereof. Jaw member 120 further includes an electrode 130. Structural frame 121 defines a proximal flange portion 126 and a distal body portion (see FIG. 10A) extending distally from proximal flange portion 126. Proximal flange portion 126 is bifurcated to define a pair of spaced-apart proximal flange portion segments that receive proximal flange 116 of structural frame 111 of jaw member 110 therebetween and define aligned apertures 127 configured for receipt of pivot 103 therethrough to pivotably couple jaw members 110, 120 with one another.

Jaw housing 122 of jaw member 120 is disposed about the distal body portion of structural frame 121, e.g., via overmolding, adhesion, mechanical engagement, etc., and supports tissue treating plate 123 thereon, e.g., via overmolding, adhesion, mechanical engagement, depositing (such as, for example, via sputtering), etc. Tissue treating plate 123, as noted above, defines tissue treating surface 124. A longitudinally-extending slot 125 is defined through tissue treating plate 123 and is positioned to oppose insulative member 115 of jaw member 110 (FIG. 5A) in the approximated position. Slot 125 may extend through at least a portion of jaw housing 122, a jaw insert (if so provided), and/or other components of jaw member 120 to enable receipt of an electrode 130 at least partially within slot 125.

Electrode 130, more specifically, is disposed within longitudinally-extending slot 125 such that electrode 130 opposes insulative member 115 of jaw member 110 (FIG. 5A) in the approximated position. Electrode 130 may be configured to contact insulative member 115 (FIG. 5A) in the approximated position to regulate or contribute to regulation of a gap distance between tissue treating surfaces 114, 124 in the approximated position. Alternatively or additionally, one or more stop members (not shown) associated with jaw member 110 and/or jaw member 120 may be provided to regulate the gap distance between tissue treating surfaces 114, 124 in the approximated position.

Electrode 130 may be surrounded by an insulative member 128 disposed within slot 125 to electrically isolate thermal cutting element from tissue treating plate 123. Insulative member 128 may be separate from or formed as a portion of jaw housing 122 (or the jaw insert (not shown), if provided). Alternatively or additionally, electrode 130 may include an insulative coating on at least the sides thereof for similar purposes. Electrode 130 and insulative member 128 may similarly or differently be substantially (within manufacturing, material, and/or use tolerances) coplanar with tissue treating surface 124, may protrude from tissue treating surface 124, may be recessed relative to tissue treating surface 124, or may include different portions that are coplanar, protruding, and/or recessed relative to tissue treating surface 124. Although electrode 130 is shown engaged within jaw member 120, it is contemplated that the reverse configuration also be provided, e.g., wherein electrode 130 is engaged within jaw member 110 (FIG. 4) and insulative member 115 is engaged within jaw member 120.

In configurations where end effector assembly 100, or a portion thereof, is curved, longitudinally-extending slot 125 and electrode 130 may similarly be curved, e.g., wherein longitudinally-extending slot 125 and electrode 130 (or corresponding portions thereof) are relatively configured with reference to an arc (or arcs) of curvature rather than a longitudinal axis. Thus, the terms longitudinal, transverse, and the like as utilized herein are not limited to linear configurations, e.g., along linear axes, but apply equally to curved configurations, e.g., along arcs of curvature. In such curved configurations, insulating member 115 of jaw member 110 (FIG. 5A) is likewise curved.

Generally referring to FIGS. 1-5B, tissue treating plates 113, 123 are formed from an electrically conductive material, e.g., for conducting electrical energy therebetween for treating tissue, although tissue treating plates 113, 123 may alternatively be configured to conduct any suitable energy, e.g., thermal, microwave, light, ultrasonic, etc., through tissue grasped therebetween for energy-based tissue treatment. As mentioned above, tissue treating plates 113, 123 are coupled to activation switch 80 and electrosurgical generator “G” (FIG. 1) such that energy may be selectively supplied to tissue treating plates 113, 123 and conducted therebetween and through tissue disposed between jaw members 110, 120 to treat tissue, e.g., seal tissue on either side and extending across electrode 130. That is, in aspects, electrosurgical generator “G” (FIG. 1) is configured to energize tissue treating plates 113, 123 with Radio Frequency (RF) electrosurgical energy in a bipolar configuration wherein tissue treating plate 113 is energized to a first potential and tissue treating plate 123 is energized to a second, different potential to establish a potential gradient for energy to flow between tissue treating plates 113, 123 and through tissue grasped therebetween to treat, e.g., seal tissue.

Electrode 130, on the other hand, is configured to connect to electrosurgical generator “G” (FIG. 1) and second activation switch 90 to enable selective activation of the supply of energy to electrode 130 for treating tissue, e.g., cutting, dissecting, spot coagulating, scoring, performing an otomy, etc., that is disposed between jaw members 110, 120 (e.g., to cut previously sealed tissue into first and second sealed tissue portions, to perform scissor-like tissue cutting, etc.), positioned distally of jaw members 110, 120, or otherwise in the vicinity of electrode 130. Electrode 130 may be energized with RF electrosurgical energy in a first configuration wherein electrosurgical generator “G” (FIG. 1) energizes electrode 130 as the active electrode configured to deliver energy to tissue in contact therewith and wherein a separate return device (not shown) (e.g., a remote return pad, a separate return instrument such as a tenaculum or probe, etc.) acts as the return electrode to collect the energy for return to electrosurgical generator “G” (FIG. 1) to complete the circuit. Alternatively or additionally, electrode 130 may be energized in a second configuration wherein electrosurgical generator “G” (FIG. 1) energizes electrode 130 as the active electrode and a local return disposed on or within end effector assembly 100 acts as the return electrode to collect the energy for return to electrosurgical generator “G” (FIG. 1) to complete the circuit. For example, the local return electrode may include either or both of tissue treating plates 113, 123 and/or other portion of either or both jaw members 110, 120 such as, for example, a return wire, a return plate, a conductive surface of either or both jaw members 110, 120, or any other suitable return conductor or portion.

Various configurations of electrode 130 and/or other aspects of jaw members 110, 120 configured to facilitate tissue treatment with electrode 130 are detailed below. To the extent consistent, any or all of these features may be used with one another or in any suitable combination. Further, although described herein with reference to end effector assembly 100, the aspects and features detailed below are applicable for use with other suitable end effectors and instruments.

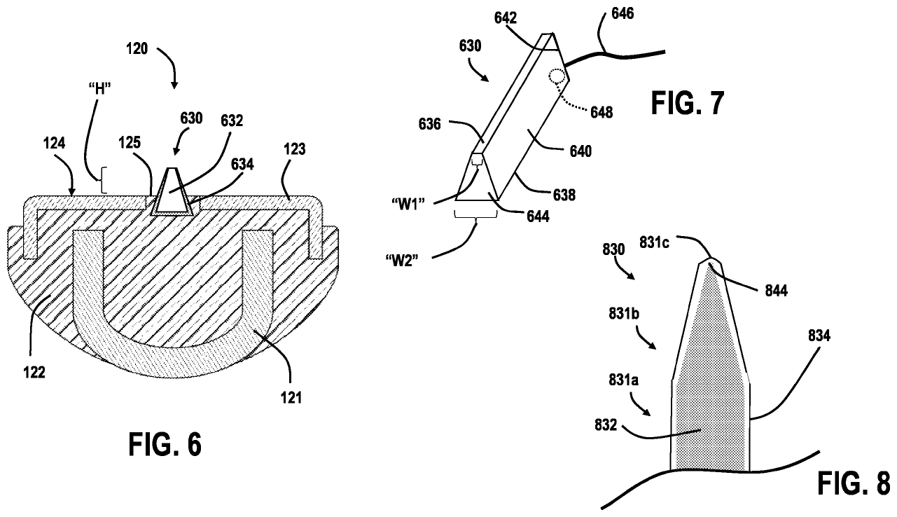

Turning to FIGS. 6 and 7, electrode 630 is shown engaged within jaw member 120, although electrode 630 may alternatively be engaged within jaw member 110 (FIG. 4). Electrode 630, to the extent consistent, may include any of the features of electrode 130 (FIG. 4), any of the other electrodes detailed herein, combinations thereof, etc. Electrode 630 includes an electrically conductive element 632 and an electrically insulative coating 634 surrounding a portion of electrically conductive element 632. Electrically conductive element 632 may be formed from stainless steel, titanium, or other suitable electrically conductive material and may be formed via etching, grinding, or in any other suitable manner. Electrically conductive element 632 includes an elongate base surface 638, an elongate tissue treating surface 636 opposing elongate base surface 638, a pair of elongate side surfaces 640, a proximal end portion 642, and a distal end portion 644. In one aspect, electrically conductive element 632 defines an elongate triangular prism shaped configuration (or an elongate triangular prism disposed on an elongate rectangular prism) except that an elongate apex of the elongate triangular prism defines the elongate tissue treating surface 636 rather than an elongate edge. In this and other aspects, elongate base surface 638 may define a relatively broad width as compared to a relatively narrow width of elongate tissue treating surface 636. In other aspects, both surfaces 636, 638 may define relatively narrow width configurations or similar or different widths. Other suitable shapes and configurations for electrically conductive element 632 are also contemplated, including configurations wherein some or all of surfaces 636, 638, and 640 include curvature, angles, steps, and/or other features.

Electrically insulative coating 634 is disposed on elongate base surface 638, elongate angled side surfaces 640, proximal end portion 642, and distal end portion 644 of electrically conductive element 632. However, elongate tissue treating surface 636 of electrically conductive element 632 remains exposed (e.g., is not coated or is only nominally coated with electrically insulative coating 634) such that electrode 630 is configured to direct a majority of the electrosurgical energy supplied thereto to elongate tissue treating surface 636 to facilitate treating tissue therewith. In aspects, electrode 630 defines a width “W1” along exposed elongate tissue treating surface 636 of from about 0.0005 inches to about 0.003 inches; in other aspects, from about 0.001 inches to about 0.002 inches. The width “W1” along elongate tissue treating surface 636 is selected to balance the advantage of providing a narrower width so as to facilitate concentration of energy at elongate tissue treating surface 636, thus requiring less power and improving electrical cutting performance, with the necessity to providing a broader width to reduce mechanical sharpness and inhibit mechanical cutting. In aspects, electrically conductive element 632 is configured such that the concentration of energy at elongate tissue treating surface 636 generates a plasma at elongate tissue treating surface 636 to facilitate cutting tissue in contact with elongate tissue treating surface 636, e.g., via plasma-assisted RF tissue cutting.

Electrode 630 may further define a width “W2” along elongate base surface 638 (including any thickness due to electrically insulative coating 634 on elongate side surfaces 640) in aspects, of from about 0.002 inches to about 0.010 inches; in other aspects, from about 0.003 inches to about 0.008 inches. In other aspects, width “W2” (including any thickness due to electrically insulative coating 634 on elongate side surfaces 640) is from about 0.016 inches to about 0.020 inches; or about 0.018 inches. In aspects, a ratio of the width of electrode 630 along elongate base surface 638 to a width of electrode 630 along elongate tissue treating surface 636 may be from about 8:1 to about 2:1; in aspects, from about 5:1 to about 3:1. In aspects, elongate side surfaces 640 are substantially planar and angled such that electrically conductive element 632 defines a smooth taper from elongate base surface 638 to elongate tissue treating surface 636, although other configurations are also contemplated including curved (convex or concave), stepped, multi-angle, combinations thereof, etc. elongate side surfaces 640. Further, in addition or as an alternative to a taper in a width of electrically conductive element 632 from elongate base surface 638 to elongate tissue treating surface 636, the thickness of electrically insulative coating 634 may likewise taper from elongate base surface 638 to elongate tissue treating surface 636, at a similar rate as the taper of electrically conductive element 632 or at a different rate. In aspects, the thickness of electrically insulative coating 634 (on each side of electrically conductive element 632) is from about 0.002 inches to about 0.006 inches at elongate base surface 638 of electrically conductive element 632 and tapers to no coating (or nominal coating) at elongate tissue treating surface 636 of electrically conductive element 632. In other aspects, the thickness of electrically insulative coating 634 (on each side of electrically conductive element 632) is about 0.004 inches at elongate base surface 638.

Electrically insulative coating 634 may be formed from glass. In other aspects, electrically insulative coating 634 is formed from ceramic, polyamide, TPE, polyphthalamide (PPA), polyetheretherketone (PEEK), polybenzimidazole (PBI), polytetrafluoroethylene (PTFE), silicone, or other suitable material. Electrically insulative coating 634 may be coated onto electrically conductive element 632 via spraying, dip coating, molding, or in any other suitable manner. In aspects, electrically insulative coating 634 may be provided via multiple, e.g., two or more, coating layers of similar or different materials. Electrically insulative coating 634, regardless of the particular configuration, provides dielectric strength of, in aspects, at least 400 Volts; in other aspects, at least 600 Volts; in still other aspects, at least 800 Volts; and in yet other aspects, at least 1000 Volts.

As shown in FIG. 7, an electrical lead wire 646 is electrically connected to proximal end portion 642 of electrically conductive element 632 or to elongate base surface 638 of electrically conductive element 632 towards proximal end portion 642 of electrically conductive element 632 to enable the supply of electrosurgical energy to electrically conductive element 632 to energize electrode 630. Electrical lead wire 646 may be soldered or otherwise attached to an exposed portion 648 of electrically conductive element 632. In order to provide exposed portion 648 of electrically conductive element 632 to enable attachment of electrical lead wire 646 thereto, exposed portion 648 of electrically conductive element 632 may be masked during application of electrically insulative coating 634 such that exposed portion 648 is not coated with electrically insulative coating 634 and, thus, remains exposed for connection of electrical lead wire 646 thereto. Alternatively, electrically insulative coating 634 may be cleaved to provide access to exposed portion 648 of electrically conductive element 632 for connection of electrical lead wire 646 thereto. Other suitable manners of attaching electrical lead wire 646 are also contemplated, including the use of intermediate structures such as, for example, tabs, contact pads, etc. male/female electrical connectors, etc.

The RF electrosurgical energy delivered to electrode 630 may be an energy waveform having a maximum peak-to-peak voltage of up to about 1000V and a root mean squared voltage (VRms) of up to about 360V. The waveform may be unpulsed or, in other aspects, may be pulsed. As noted above, the energy supplied, tougher with the configuration of electrode 630, may be such that a plasma is generated to facilitate tissue cutting (or other suitable tissue treatment)

Returning to FIG. 6, a portion of electrode 630 is engaged within jaw member 120. Electrode 630 may be engaged within jaw member 120 via capturing a portion of electrode 630 with one or more overmolds, e.g., of jaw housing 122, a jaw insert (not shown), etc., or may be engaged within jaw member 120 in any other suitable manner (such as, for example, detailed above with respect to electrode 130 (FIG. 4)). Regardless of the manner of engagement of electrode 630 within jaw member 120, electrode 630 is positioned to extend a height “H” from tissue treating surface 124 of tissue treating plate 123 of jaw member 120 towards jaw member 110. Height “H” is selected to balance limiting the height electrode 630 extends from tissue treating surface 124 to inhibit the reduction of jaw grasping force applied to tissue grasped between tissue treating surfaces 114, 124 of jaw members 110, 120, respectively (see FIG. 4), with increasing the height electrode 630 extends from tissue treating surface 124 to increase tissue treating performance, e.g., tissue cutting, using electrode 630. In aspects, height “H” is from about 0.010 inches to about 0.025 inches; in other aspects, from about 0.015 inches to about 0.025 inches. In aspects, height “H” varies along at least a portion of the length of tissue treating surface 124 of tissue treating plate 123 of jaw member 120. For example, height “H” may taper in the distal to proximal direction, in the proximal to distal direction, from one or both ends towards the center, or from the center towards one or both ends. This taper may follow an angled plane, a curvature, one or more steps, or any other suitable configuration. In aspects where a taper is provided, the above-noted height ranges may be average heights, maximum heights, or minimum heights.

Turning to FIG. 8, electrode 830 includes an electrically conductive element 832 and an electrically insulative coating 834. Electrode 830, to the extent consistent, may include any of the features of electrode 130 (FIG. 4), electrode 630 (FIGS. 6 and 7), any of the other electrodes detailed herein, combinations thereof, etc. Electrode 830 includes a body portion 831a and a tapered distal portion 831b. Tapered distal portion 831b of electrode 830 narrows in width from body portion 831a to distal tip 831c of electrode 830. Distal face 844 of electrically conductive element 832 defines a substantially planar configuration having a width of from about 0.0005 inches to about 0.0020 inches; or, in other aspects, from about 0.0005 inches to about 0.0010 inches. In aspects, distal face 844 is omitted and electrically conductive element 832 terminates at an edge at distal tip 831c of electrode 830. Such a narrow distal face 844 (or distal edge) facilitates open dissection, performing otomies, and other electrical tissue treating (e.g., cutting), procedures. Other configurations are also contemplated.

In aspects, electrically insulative coating 834 surrounds distal face 844. Further, a thickness of electrically insulative coating 834 about tapered distal portion 831b, e.g., extending from body portion 831a to distal tip 831c, may be substantially constant or varied (such as, for example, tapering similarly as the taller of the width of electrode 830).

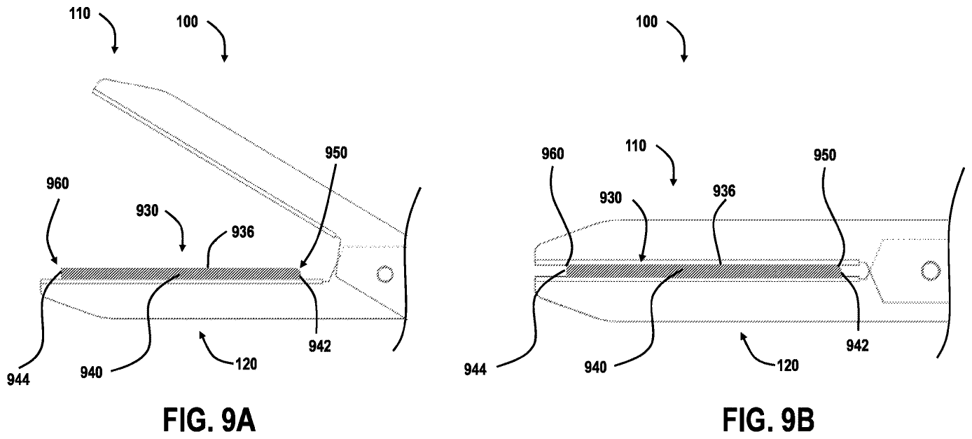

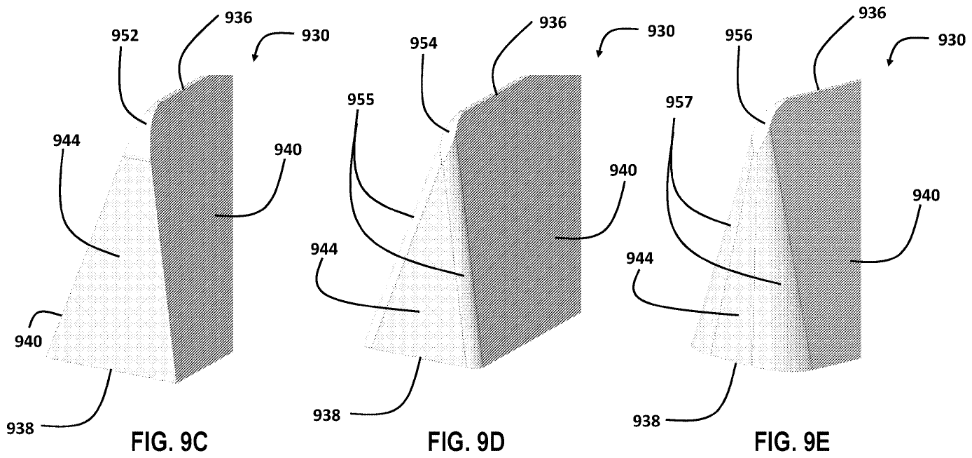

With reference to FIGS. 9A and 9B, end effector assembly 100 is shown including electrode 930 engaged within jaw member 120 (e.g., similarly as detailed hereinabove with respect to electrode 630 (FIG. 6)). Electrode 930, to the extent consistent, may include any of the features of electrode 130 (FIG. 4), electrode 630 (FIGS. 6 and 7), any of the other electrodes detailed herein, combinations thereof, etc.

Electrode 930 is configured to enhance coating insulation and reduce current concentrations and includes an elongate upper extent 936 (e.g., an upper surface or upper edge), an elongate base extent 938 (FIGS. 9C-9E; e.g., a base surface or edge), a pair of elongate side surfaces 940 (only one of which is shown in FIGS. 9A and 9B), a proximal extent 942 (e.g., a proximal face or proximal edge), and a distal extent 944 (e.g., a distal face or distal edge). A first transition 950 is defined between elongate upper extent 936 and proximal extent 942 and a second transition 960 is defined between elongate upper extent 936 and distal extent 944. First and second transitions 950, 960 may be configured similar to one another or different from one another. In aspects, first and second transitions 950, 960 define radii of curvature. For example, in aspects, first transition 950 may define a first radius of curvature and second transition 960 may define a second radius of curvature. The first radius of curvature may be greater than the second radius of curvature such that first transition 950 defines a more gradual, curved, or pronounced transition compared to second transition 960. In aspects, a ratio of the first radius of curvature to the second radius of curvature may be, for example, from about 1.5:1 to about 6:1, in other aspects, from about 2:1 to about 4:1. The opposite configuration is also contemplated, as is a configuration wherein the radii of curvature are substantially similar. Other suitable geometries for the configuration of electrode 930 to enhance coating insulation and reduce current concentrations are also contemplated.

Rather than both transitions 950, 960 defining radii of curvature, first and second transitions 950, 960 may define beveled surfaces having similar or different lengths. For example, in aspects, first transition 950 may define a beveled surface having a first length while second transition 960 defines a beveled surface having a second, shorter length. The opposite configuration is also contemplated.

Further still, first transition 950 may define a first configuration, e.g., one of a radius of curvature or a beveled surface, while second transition 960 defines a second configuration different from the first configuration, e.g., the other of a radius of curvature or a beveled surface.

The above-detailed configuration of first transition 950 inhibits mechanical cutting by electrode 930 as first and second jaw members 110, 120 are moved from the spaced-apart position towards the approximated position to grasp tissue therebetween, minimizes energy concentration at first transition 950, and inhibits erosion at first transition 950. Additionally or alternatively, the above-detailed configuration of second transition 960 inhibits mechanical cutting by electrode 930, minimizes energy concentration at second transition 960, and inhibits erosion at second transition 960. The positioning of first and second transitions 950, 960 along jaw member 120 may warrant different configurations thereof, as noted above. As an example, since second transition 960 is further from pivot 103 as compared to first transition 950 and faces away from pivot 103 while first transition faces towards pivot 103, mechanical cutting during approximation of jaw members 110, 120 at first transition 950 may be a greater risk than at second transition 960 and, thus, second transition 960 need not be as gradual, curved, or pronounced as first transition 950. As a result, second transition 960 may be configured (and positioned) to facilitate tissue treatment, e.g., open dissection, performing otomies, etc. In other aspects, the first and second transitions 950, 960 may define the same configuration.

Referring to FIGS. 9C-9E, various transition configurations 952, 954, 956 in accordance with the present disclosure as shown. Although shown at the distal end of electrode 930, transition configurations 952, 954, 956 are configured for use as the first and/or second transitions 950, 960 of electrode 930 (FIGS. 9A and 9B) at the proximal and/or distal ends of electrode 930.

With reference to FIG. 9C, transition configuration 952 defines a convex, curved surface extending between elongate upper extent 936 and distal extent 944 of electrode 930, thus eliminating an edge at the interface between elongate upper extent 936 and distal extent 944. The convex, curved surface of transition configuration 952 extends in the width direction between the edges formed at the interfaces between distal extent 944 and each of the elongate side surfaces 940. The convex, curved surface of transition configuration 952 thus increases in width as it extends from elongate upper extent 936 to distal extent 944 and towards elongate base extent 938 due to the fact that electrode 930 increases in width from elongate upper extent 936 towards elongate base extent 938.

As shown in FIG. 9D, transition configuration 954 defines a convex, curved surface extending between elongate upper extent 936 and distal extent 944 of electrode 930, thus eliminating an edge at the interface between elongate upper extent 936 and distal extent 944. Further, elongated transitions 955, e.g., radiused surfaces or other suitable transitions, are defined at the interfaces between distal extent 944 and each of the elongate side surfaces 940. Elongated transitions 955 are substantially constant along the heights of the interfaces between distal extent 944 and each of the elongate side surfaces 940. In this configuration, rather than the convex, curved surface of transition configuration 954 increasing in width as it extends from elongate upper extent 936 to distal extent 944 and towards elongate base extent 938, the convex, curved surface of transition configuration 954 defines a relatively wider intermediate portion and relatively narrower end portions.

Referring to FIG. 9E, transition configuration 956 is similar to transition configuration 954 (FIG. 9D) except that elongated transitions 957 defined at the interfaces between distal extent 944 and each of the elongate side surfaces 940 define increasing radii of curvature from elongate upper extent 936 towards elongate base extent 938, thus defining a conical-like distal portion of electrode 930.

The above-detailed transition configurations inhibit mechanical cutting with electrode 930 and erosion at the interfaces between different surfaces of electrode 930, thus improving performance. Other suitable transition configurations are also contemplated.

Turning to FIGS. 10A and 10B, in aspects, as noted above, jaw member 110 may include a longitudinally-extending insulative member 1015 extending along at least a portion of the length of tissue treating surface 114, e.g., within a channel defined longitudinally through tissue treating plate 113. Insulative member 1015 is positioned to oppose electrode 130 of jaw member 120 in the approximated position of jaw members 110, 120. In aspects, an exposed surface of insulative member 1015 is substantially co-planar with tissue treating surface 114. In other aspects, the exposed surface of insulative member 1015 protrudes from or is recessed relative to tissue treating surface 114. The extent to which insulative member 1015 protrudes, is recessed, or is co-planar may vary along the length of tissue treating surface 114, similarly as detailed above with respect to the tapering in height of electrode 630 (FIG. 6).

Insulative member 1015 is at least partially compliant such that insulative member 1015 at least partially resiliently compresses upon urging of electrode 130 into contact insulative member 1015 as jaw members 110, 120 are moved to the approximated position. The materials and configuration of insulative member 1015 are selected to inhibit the reduction of jaw grasping force applied to tissue grasped between tissue treating surfaces 114, 124 of jaw members 110, 120, respectively, due to the presence of electrode 130 and to facilitate tensioning tissue disposed between electrode 130 and insulative member 1015 to facilitate electrical cutting of tissue grasped between jaw members 110, 120 using electrode 130. In aspects, insulative member 1015 defines a durometer of from about 55 D to about 65 D; in other aspects, from about 58 D to about 62 D.

In aspects, insulative member 1015 may define a rectangular prism-shaped configuration defining a width “W3” and a depth “D.” Width “W3,” in aspects, may define a maximum and/or average of from about 0.025 inches to about 0.095 inches; in other aspects, from about 0.030 inches to about 0.090 inches. Insulative member 1015 may defines a tapering width “W3” in the proximal to distal direction. In such aspects, the maximum of width “W3,” e.g., at the proximal end of tissue treating surfaces 114, may be from about 0.075 inches to about 0.095 inches; in other aspects, from about 0.080 inches to about 0.090 inches. The midpoint of width “W3,” e.g., at a midpoint along tissue treating surfaces 114, may be, in aspects, from about 0.025 inches to about 0.045 inches; in other aspects, from about 0.030 inches to about 0.040 inches. The minimum of width “W3” (wherein the opposing side edges of insulative member 1015 define an angle of about 90 degrees), e.g., at the distal end of insulative member 1015, may be from about 0.008 inches to about 0.018 inches; in other aspects, from about 0.011 inches to about 0.015 inches. In aspects, the difference between the maximum and minimum of width “W3,” e.g., at the proximal an distal ends of insulative member 1015, may be, in aspects, from about 0.060 inches to about 0.085 inches. In aspects, the maximum width, minimum width, and/or average width may be provided in accordance with the above-noted width ranges. Other suitable shapes and/or dimensions of insulative member 1015 are also contemplated.

The depth “D” of insulative member 1015 may be from about 0.03 inches to about 0.05 inches; in other aspects, from about 0.035 inches to about 0.045 inches. Larger depths “D” are also contemplated. Further, in aspects, the depth “D” may taper in the bottom to top direction (e.g., towards jaw member 120), wherein the depth “D” at the bottom of insulative member 1015 is between, for example, about 0.005 to about 0.015 inches greater than the depth “D” at the top of insulative member 1015. In such aspects, the maximum depth, minimum depth, and/or average depth may be provided in accordance with the above-noted width ranges.

Aspects of this disclosure may be further described by reference to the following numbered paragraphs:
1. An end effector assembly of a surgical instrument, the end effector assembly comprising: first and second jaw members each defining a tissue treating surface, at least one of the first jaw member or the second jaw member movable relative to another of the first jaw member or the second jaw member from a spaced-apart position to an approximated position to grasp tissue between the tissue treating surfaces of the first and second jaw members; and an electrode supported by the second jaw member, the electrode extending longitudinally along at least a portion of a length of the second jaw member and protruding from the second jaw member towards the first jaw member, the electrode including an electrically conductive element and an electrically insulative coating, the electrically conductive element defining an elongate base surface, an elongate tissue treating surface opposing the elongate base surface, first and second elongate side surfaces, a proximal end portion, and a distal end portion, wherein the electrically insulative coating covers the elongate base surface, the first and second elongate side surfaces, the proximal end portion, and the distal end portion, leaving the elongate tissue treating surface exposed, and wherein the electrically conductive element is adapted to connect to a source of Radio Frequency (RF) energy for energizing the electrically conductive element with RF energy and concentrating the RF energy at the exposed elongate tissue treating surface.
2. The end effector assembly according to paragraph 1, wherein the electrode defines a width at the elongate tissue treating surface of from about 0.001 inches to about 0.002 inches.
3. The end effector assembly according to paragraph 1 or 2, wherein a thickness of the electrically insulative coating on each of the first and second elongate side surfaces tapers in a direction from the elongate base surface to the elongate tissue treating surface.
4. The end effector assembly according to paragraph 3, wherein a maximum thickness of the electrically insulative coating on each of the first and second elongate side surfaces is from about 0.002 inches to about 0.006 inches.
5. The end effector assembly according to any preceding paragraph, wherein the electrode protrudes from the tissue treating surface of the second jaw member towards the first jaw member a height of from about 0.018 inches to about 0.024 inches.
6. The end effector assembly according to any preceding paragraph, wherein the electrode includes an exposed portion of the electrically conductive element at or towards the proximal end portion of the electrically conductive element for connection of an electrical lead wire to the electrically conductive element to provide the RF energy to the electrically conductive element.
7. The end effector assembly according to any preceding paragraph, wherein the electrode includes a body portion and a tapered distal portion extending distally from the body portion to a distal tip of the electrode.
8. The end effector assembly according to any preceding paragraph, wherein the electrode tapers in width along at least a portion of a height of the electrode extending from the elongate base surface to the elongate tissue treating surface.
9. The end effector assembly according to paragraph 8, wherein the first and second elongate side surfaces are angled inwardly towards one another to define the taper.
10. The end effector assembly according to any preceding paragraph, wherein the electrode defines a transition at an interface between the elongate tissue treating surface and at least one of the proximal end portion or the distal end portion.
11. The end effector assembly according to any preceding paragraph, wherein the electrode defines a first transition at an interface between the elongate tissue treating surface and the proximal end portion, and a second transition at an interface between the elongate tissue treating surface and the distal end portion.
12. The end effector assembly according to paragraph 11, wherein the first transition is more gradual than the second transition.
13. The end effector assembly according to paragraph 11 or 12, wherein the first transition defines a first radius of curvature and wherein the second transition defines a second radius of curvature less than the first radius of curvature.
14. The end effector assembly according to any preceding paragraph, further comprising a compliant electrically insulative member disposed within the first jaw member and positioned to oppose the electrode in the approximated position.
15. The end effector assembly according to paragraph 14, wherein the compliant electrically insulative member defines a durometer of from about 55 D to about 65 D.
16. The end effector assembly according to any preceding paragraph, wherein the tissue treating surfaces of the first and second jaw members are adapted to connect to a source of RF energy for energizing the tissue treating surfaces of the first and second jaw members with RF energy at different potentials.
17. The end effector assembly according to any preceding paragraph, wherein one of the first jaw member or the second jaw member is fixed and wherein another of the first jaw member or the second jaw member is movable relative to the fixed jaw member.
18. The end effector assembly according to any preceding paragraph, wherein the electrically insulative coating is glass.
19. An end effector assembly of a surgical instrument, the end effector assembly comprising: first and second jaw members each defining a tissue treating surface, at least one of the first jaw member or the second jaw member movable relative to another of the first jaw member or the second jaw member from a spaced-apart position to an approximate position to grasp tissue between the tissue treating surfaces of the first and second jaw members; and an electrode supported by the second jaw member, the electrode extending longitudinally along at least a portion of a length of the second jaw member between proximal and distal ends of the electrode and protruding from the second jaw member towards the first jaw member to an elongate upper extent of the electrode, the electrode adapted to connect to a source of Radio Frequency (RF) energy for energizing the electrode with RF energy, wherein the electrode defines a first transition between the elongate upper extent of the electrode and the proximal end of the electrode and a second transition between the elongate upper extent of the electrode and the distal end of the electrode.
20. The end effector assembly according to paragraph 19, wherein the first transition is more gradual than the second transition.
21. The end effector assembly according to paragraph 19 or 20, wherein the first transition defines a first radius of curvature and wherein the second transition defines a second radius of curvature less than the first radius of curvature.
22. The end effector assembly according to any one of paragraphs 19-21, wherein the electrode includes an electrically conductive element and an electrically insulative coating, the electrically insulative coating substantially covering the electrically conductive element except along the elongate upper extent of the electrode such that the electrically conductive element is exposed along the elongate upper extent of the electrode.
23. The end effector assembly according to any one of paragraphs 19-22, wherein the tissue treating surfaces of the first and second jaw members are adapted to connect to a source of RF energy for energizing the tissue treating surfaces of the first and second jaw members with RF energy at different potentials.
24. The end effector assembly according to any one of paragraphs 19-23, wherein one of the first jaw member or the second jaw member is fixed and wherein another of the first jaw member or the second jaw member is movable relative to the fixed jaw member.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

1. An end effector assembly of a surgical instrument, the end effector assembly comprising:
first and second jaw members each defining a tissue treating surface, at least one of the first jaw member or the second jaw member movable relative to another of the first jaw member or the second jaw member from a spaced-apart position to an approximated position to grasp tissue between the tissue treating surfaces of the first and second jaw members; and
an electrode supported by the second jaw member, the electrode extending longitudinally along at least a portion of a length of the second jaw member and protruding from the second jaw member towards the first jaw member, the electrode including an electrically conductive element and an electrically insulative coating, the electrically conductive element defining an elongate base surface, an elongate tissue treating surface opposing the elongate base surface, first and second elongate side surfaces, a proximal end portion, and a distal end portion,
wherein the electrically insulative coating covers the elongate base surface, the first and second elongate side surfaces, the proximal end portion, and the distal end portion, leaving the elongate tissue treating surface exposed, and
wherein the electrically conductive element is adapted to connect to a source of Radio Frequency (RF) energy for energizing the electrically conductive element with RF energy and concentrating the RF energy at the exposed elongate tissue treating surface.

2. The end effector assembly according to claim 1, wherein the electrode defines a width at the elongate tissue treating surface of from about 0.001 inches to about 0.002 inches.

3. The end effector assembly according to claim 1, wherein a thickness of the electrically insulative coating on each of the first and second elongate side surfaces tapers in a direction from the elongate base surface to the elongate tissue treating surface.

4. The end effector assembly according to claim 3, wherein a maximum thickness of the electrically insulative coating on each of the first and second elongate side surfaces is from about 0.002 inches to about 0.006 inches.

5. The end effector assembly according to claim 1, wherein the electrode protrudes from the tissue treating surface of the second jaw member towards the first jaw member a height of from about 0.018 inches to about 0.024 inches.

6. The end effector assembly according to claim 1, wherein the electrode includes an exposed portion of the electrically conductive element at or towards the proximal end portion of the electrically conductive element for connection of an electrical lead wire to the electrically conductive element to provide the RF energy to the electrically conductive element.

7. The end effector assembly according to claim 1, wherein the electrode includes a body portion and a tapered distal portion extending distally from the body portion to a distal tip of the electrode.

8. The end effector assembly according to claim 7, wherein the first and second elongate side surfaces are angled inwardly towards one another to define the taper.

9. The end effector assembly according to claim 1, wherein the electrode defines a transition at an interface between the elongate tissue treating surface and at least one of the proximal end portion or the distal end portion.

10. The end effector assembly according to claim 1, wherein the electrode defines a first transition at an interface between the elongate tissue treating surface and the proximal end portion, and a second transition at an interface between the elongate tissue treating surface and the distal end portion, wherein the first transition is more gradual than the second transition.

11. The end effector assembly according to claim 1, further comprising a compliant electrically insulative member disposed within the first jaw member and positioned to oppose the electrode in the approximated position.

12. The end effector assembly according to claim 11, wherein the compliant electrically insulative member defines a durometer of from about 55 D to about 65 D.

13. The end effector assembly according to claim 1, wherein the tissue treating surfaces of the first member and second jaw members are adapted to connect to a source of RF energy for energizing the tissue treating surfaces of the first and second jaw members with RF energy at different potentials.

14. The end effector assembly according to claim 1, wherein one of the first jaw member or the second jaw member is fixed and wherein another of the first jaw member or the second jaw member is movable relative to the fixed jaw member.

15. The end effector assembly according to claim 1, wherein the electrically insulative coating is glass.
